(19) United States
(12) Patent Application Publication
(10) Pub. No.: US 2024/0245389 A1
(43) Pub. Date: Jul. 25, 2024

(54) METHODS AND SYSTEMS FOR MODULAR TRANSDUCER PROBE WITH REDUCED FOOTPRINT

(71) Applicant: GE Precision Healthcare LLC, Wauwatosa, WI (US)

(72) Inventors: Warren Lee, Niskayuna, NY (US); Jean-Luc Diot, Valbonne (FR); Giandonato Stallone, Valbonne (FR); Naresh K Rao, Niskayuna, NY (US)

(21) Appl. No.: 18/158,389

(22) Filed: Jan. 23, 2023

(51) Int. Cl. A61B 8/00; B06B 1/06; H10N 39/00

(52) U.S. Cl. CPC A61B 8/4494; A61B 8/4411; B06B 1/0622; B06B 1/067; H10N 39/00; B06B 2201/76

(57) ABSTRACT

Various methods and systems are provided for an electro-acoustic module for a transducer probe. In one example, the electro-acoustic module may include an acoustic stack and at least one application-specific integrated circuit (ASIC) electrically coupled to the acoustic stack by an interconnect having a fan-out architecture. The electro-acoustic module may have an active aperture substantially equal to an overall size of the electro-acoustic module in at least one or an azimuth and an elevation direction.

### FIELD

Embodiments of the subject matter disclosed herein relate to a transducer probe for a medical device.

### BACKGROUND

Transducer probes are used in a variety of applications to convert energy from a physical form to an electrical form. For example, a transducer probe may include piezoelectric materials which generate electrical voltage from a mechanical stress or strain exerted on the materials. The piezoelectric materials may be arranged as an array of elements forming an active area of the transducer probe. In particular, ultrasound transducer probes may include integrated front-end application-specific integrated circuits (ASICs) including one or more ASICs electrically coupled to the array. In order to enable the electrical coupling, a pitch (e.g., a distance between centers of adjacent electrical contacts or bumps) of the ASICs may be similar or equal to a pitch (e.g., a distance between centers of adjacent elements) of the array. Input/output (I/O) connectors may be located peripheral to the active area of the transducer probe to allow the I/O connectors to be accessed. As a result, an overall area of the transducer probe includes an area occupied by the I/O connectors, e.g., an overhead area, in addition to the active area formed by the array of elements.

### BRIEF DESCRIPTION

In one embodiment, an electro-acoustic module comprises an acoustic stack and at least one application-specific integrated circuit (ASIC) electrically coupled to the acoustic stack by an interconnect having a fan-out architecture. The electro-acoustic module may have an active aperture substantially equal to an overall size of the electro-acoustic module in at least one or an azimuth and an elevation direction. In this way, the electro-acoustic module may be used for a variety of transducer probe types and an active area of the electro-acoustic module may be maximized to enhance a quality of data acquired by the transducer probe.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example of an acoustic stack of an ultrasound transducer.

FIG. 2 shows an example of a coupling of an acoustic stack to ASICs via a multilayer flex interconnect.

FIG. 3 shows an example of a coupling of an acoustic stack to a fan-out architecture interconnect.

FIG. 4 shows a first example of an electro-acoustic module (EAM) formed of the acoustic stack and the fan-out architecture interconnect of FIG. 3.

FIG. 5 shows a second example of an EAM incorporating a fan-out architecture interconnect.

FIG. 6 shows a third example of an EAM incorporating a fan-out architecture interconnect.

FIG. 7 shows a fourth example of an EAM incorporating a fan-out architecture interconnect.

FIG. 8 shows a fifth example of an EAM incorporating a fan-out architecture interconnect.

FIG. 9 shows a sixth example of an EAM incorporating a fan-out architecture interconnect

FIG. 10 shows a first example of a continuous array formed of tiled EAMs.

FIG. 11 shows a second example of a continuous array formed of tiled EAMs, the continuous array including a first thermal substrate configuration.

FIG. 12 shows a third example of a continuous array formed of tiled EAMs, the continuous array including a second thermal substrate configuration.

FIG. 13 shows an example of an EAM incorporating a fan-out architecture interconnect and having peripheral input/output (I/O) connections.

FIG. 14A shows an example of a first EAM having a first acoustic stack coupled to an ASIC by a first fan-out architecture interconnect.

FIG. 14B shows an example of a second EAM having a second acoustic stack coupled to the ASIC of FIG. 14A by a second fan-out architecture interconnect.

FIG. 15 shows an example of a method for assembling an EAM having a fan-out architecture interconnect.

FIG. 16 shows an example of a method for manufacturing transducer probes having a fan-out architecture interconnect and incorporating a common ASIC.

FIG. 17 shows an example of a line of ultrasound probes manufactured with a common ASIC.

FIG. 18 shows an example of a curved EAM having a fan-out architecture interconnect.

### DETAILED DESCRIPTION

A transducer probe for a medical device may include an acoustic stack formed of an array of elements for generating an acoustic signal. The acoustic stack may be coupled to a multilayer flex interconnect to enable electrical connectivity between the acoustic stack and at least one application-specific integrated circuit (ASIC), as well as to other components of the medical device via input/output (I/O) connectors. A pitch of both the multilayer flex interconnect bumps (e.g., electrical contacts formed of solder and metal) and the ASIC bumps may correspond to a pitch of the elements of the acoustic stack array in order to align the bumps of the multilayer flex interconnect and of the ASIC with each of the elements, thereby forming individual electrical circuits for each of the elements. Bumps providing I/O connectivity at the multilayer flex interconnect may be arranged outside of an active area formed defined by the array of elements, increasing an overall footprint of the transducer relative to the active area.

The matching of the pitches demands individualized fabrication of ASIC according to a specific transducer array, e.g., a specific probe type, which may be time-consuming and costly. Further, both transmitter (TX) and receiver (RX) circuits may be incorporated into the ASIC, combining both high voltage and low voltage circuits into a single chip and complicating optimization of ASIC performance. In addition, an overall area of the transducer, e.g., along a plane perpendicular to a propagation direction of the transducer array, may be larger than the active area of the transducer due to a demand for I/O connections along a top side of the multilayer flex interconnect. As a result, I/O connections may be placed peripherally around the active area, increasing a footprint of the transducer and reducing a ratio of the active area to the overall area of the transducer (e.g., relative to a ratio of 1:1). Increasing the ratio of the active area to the overall area of may be desirable to minimize acoustically inactive areas of the transducer which may increase signal generation efficiency.

The issues described above may be at least partially addressed by configuring a transducer probe with a multi-layer interposer having a fan-out architecture. Herein, the multi-layer interposer is referred to as a fan-out architecture interconnect, where the fan-out architecture is a stacking of layers with electrical connectors. The electrical connectors occupy an overall area at each layer and the overall area changes sequentially such that the overall area decreases progressively with each layer. The fan-out architecture interconnect may be used in place of a multilayer flex interconnect and may allow one or more ASICs of the transducer to have a pitch that differs from a pitch of an acoustic stack to which the one or more ASICs may be coupled. By obviating a demand for matching pitches, the transducer array may be modular and adaptable to different types of transducer probes. Additionally, the fan-out architecture allows the I/O connections to be relocated such that an overall area (e.g., an overall aperture) of the transducer array is substantially equal to the active area (e.g., an active aperture). It will be appreciated that the use of “substantially” includes a small degree of freedom in variability, such as 5%. As such, a footprint of the transducer may be reduced, allowing the transducer to be used in applications where a small transducer size is demanded.

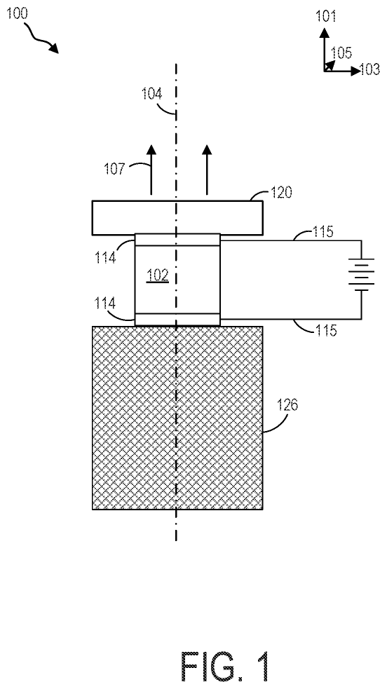

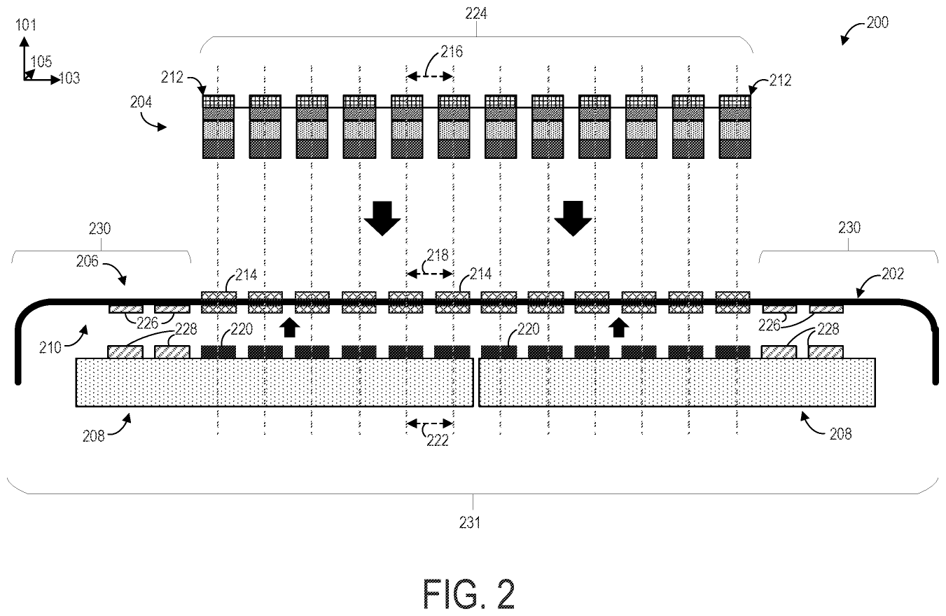

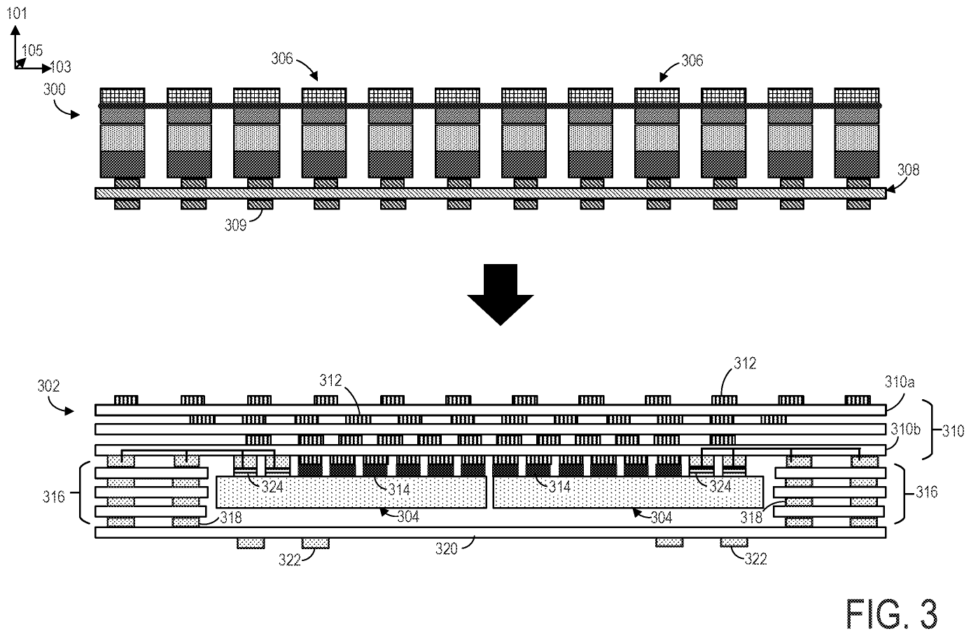

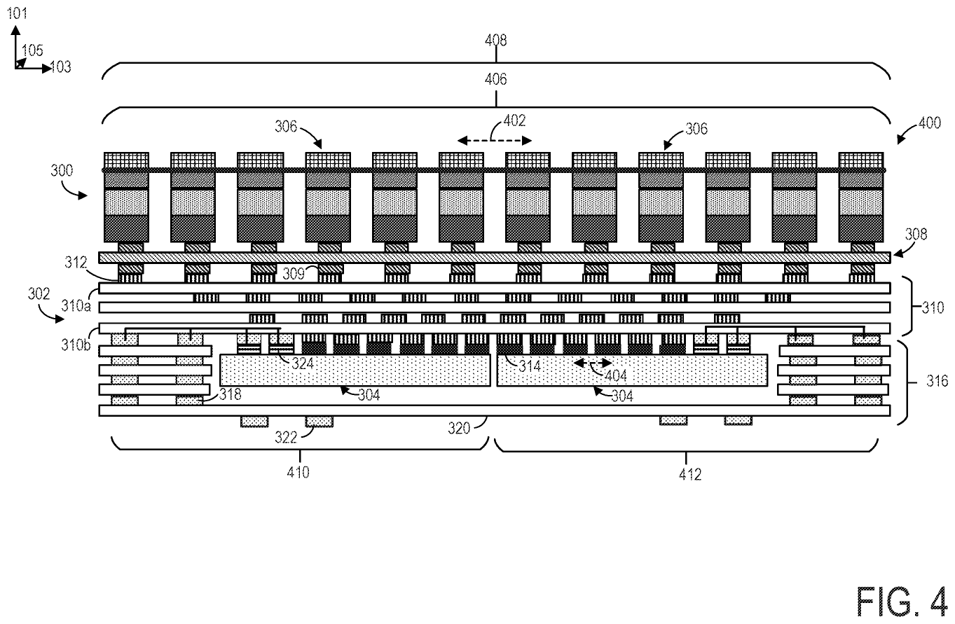

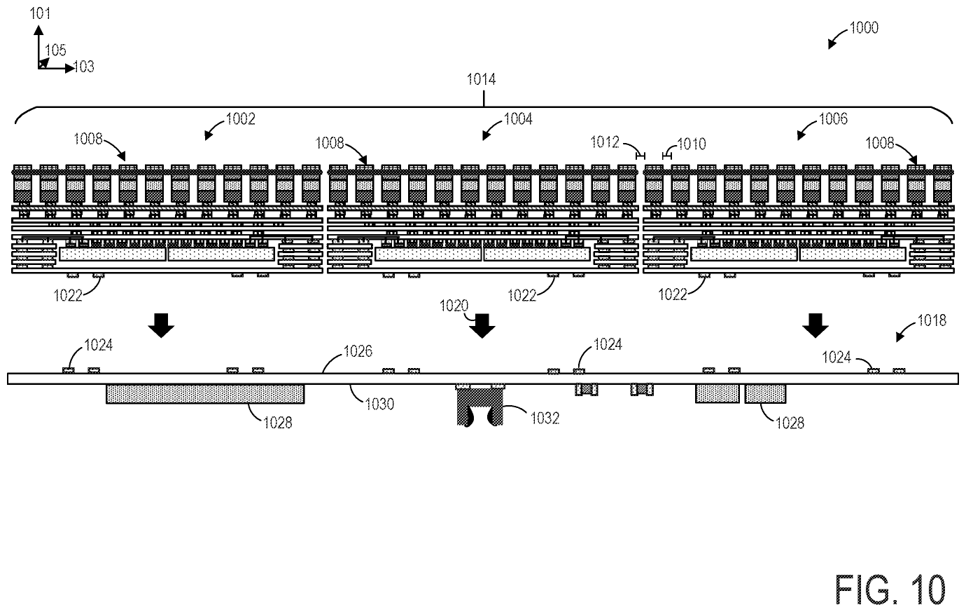

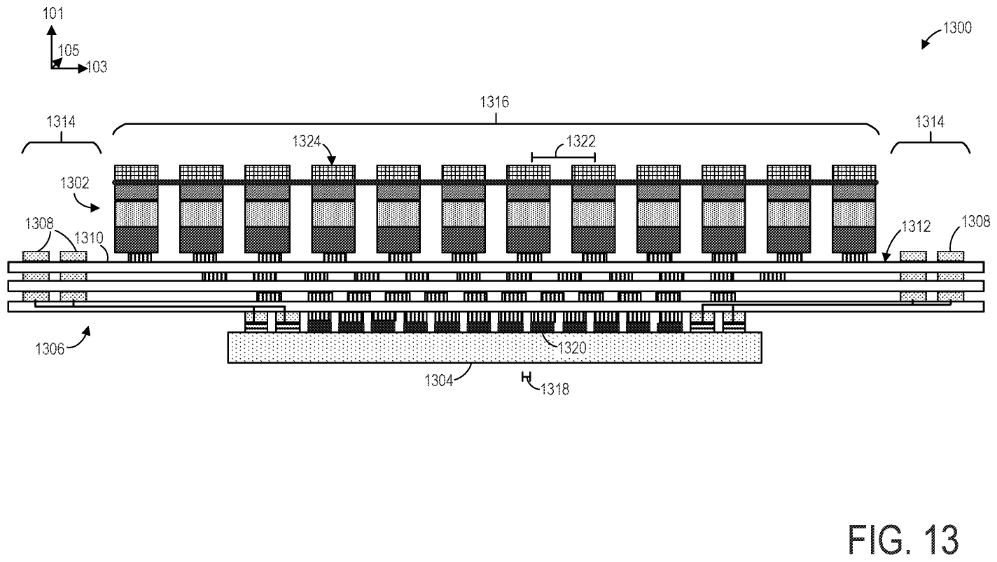

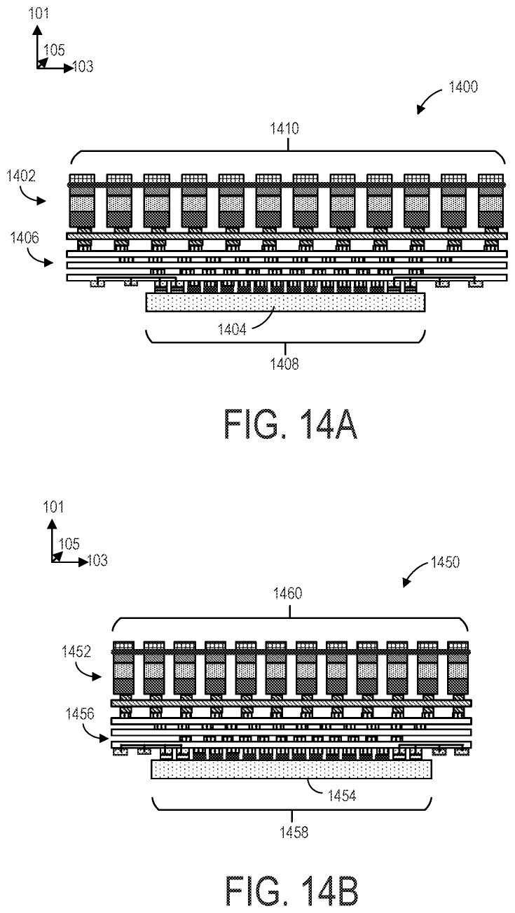

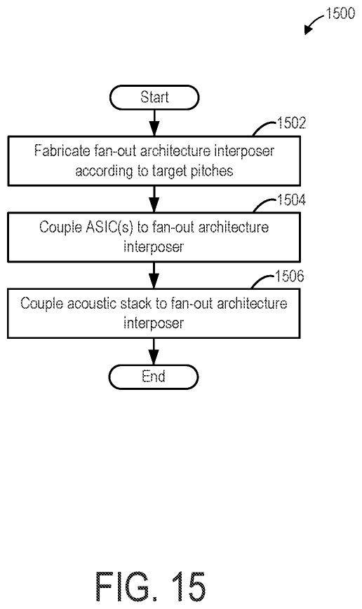

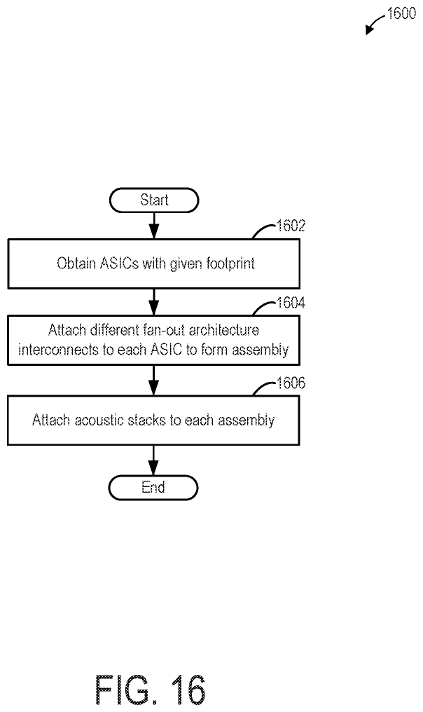

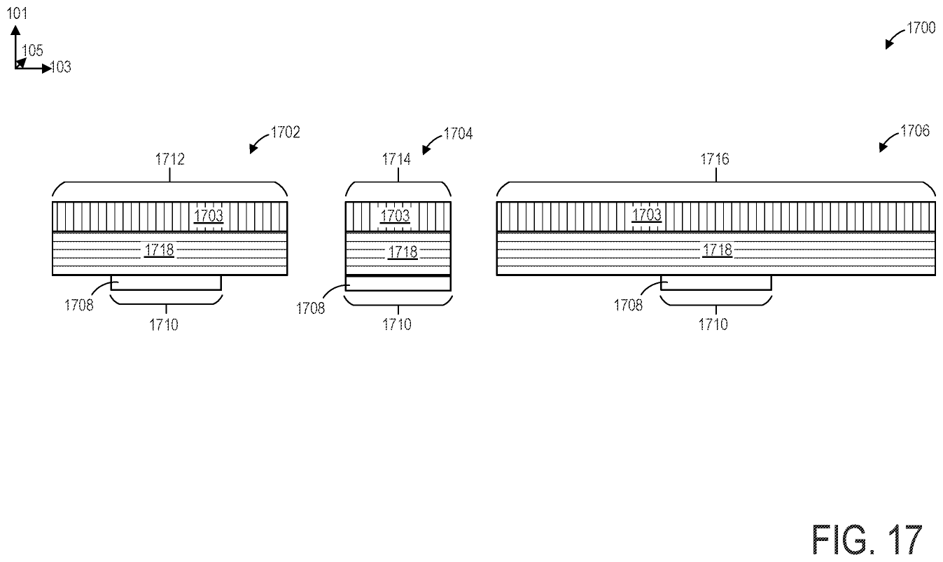

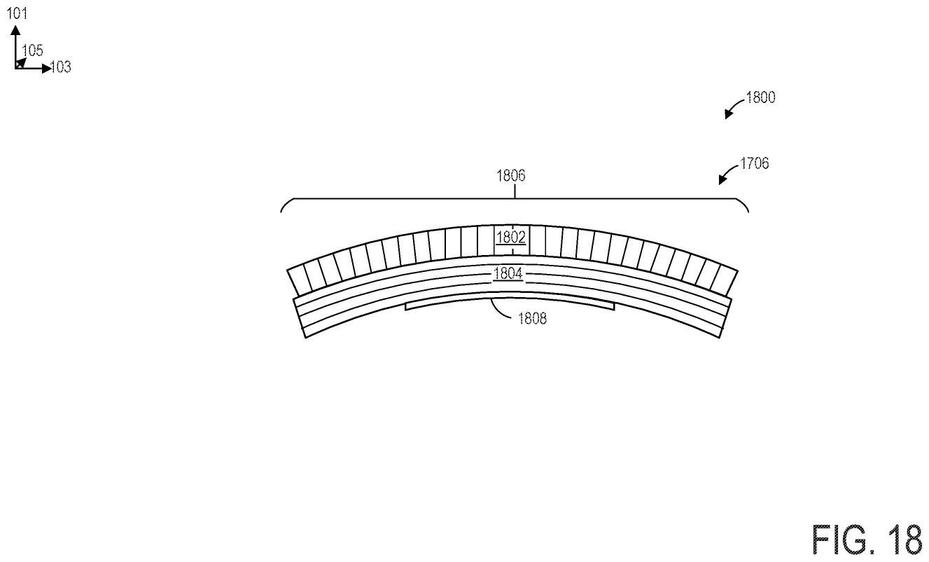

As shown in FIG. 1, a transducer may include components including an acoustic stack. In some examples, as shown herein, the transducer, e.g., transducer probe, may be configured with at least one electro-acoustic module (EAM), where the EAM includes the acoustic stack, at least one ASIC, and an interconnect coupling the acoustic stack to the ASIC. For example, advanced matrix array probes may include one or more EAMs enclosed within a probe housing, along with other probe components. The transducer may be one of a variety of transducer types, including piezoelectric transducers, micromachined ultrasound transducers (MUTs), capacitive micromachined ultrasound transducers (cMUTs), piezoelectric micromachined ultrasound transducers (pMUTs), etc. The acoustic stack may be diced into elements with a target element pitch. In a conventional transducer, the acoustic stack may be electrically coupled to ASICs via a multilayer flex interconnect, as illustrated in FIG. 2, which demands positioning of I/O connections in peripheral regions such as overhead areas. Arrangement of the I/O connections as such may increase an overall size of the transducer. By configuring the acoustic stack with a fan-out architecture interconnect, as shown in FIG. 3, ASICs with a pitch that differs from a pitch of the acoustic stack elements may be incorporated. Examples of variations in configurations of EAMs having fan-out architecture interconnects are shown in FIGS. 4-9 and examples of transducers with continuous arrays formed of tiled EAMs are depicted in FIGS. 10-12. An alternate example of an EAM having the fan-out architecture interconnect but with peripherally positioned I/O connections is illustrated in FIG. 13. The use of the fan-out architecture interconnect may enable acoustic stacks with different footprints (e.g., active apertures or areas) to be coupled to a given ASIC, as illustrated in FIGS. 14A-14B. An example of a method for assembling an EAM as a chip-scale package (CSP) with a maximized active aperture is depicted in FIG. 15 and a method for manufacturing transducer probes having the fan-out architecture interconnect and each including a common ASIC is shown in FIG. 16. The method of FIG. 16 may be used to produce a line of transducer probes, as shown in FIG. 17, having different footprints and active apertures but a common ASIC. Furthermore, by coupling an ASIC to an acoustic stack via the fan-out architecture interconnect, the fan-out architecture interconnect may be used in a curved transducer probe, as illustrated in FIG. 18.

FIGS. 1-14B, and 17-18 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

An ultrasound probe includes one or more active components for generating an ultrasonic signal. An example of an active component, or piezoelectric element 102 of an ultrasound probe is shown in a schematic diagram of an acoustic stack 100 in FIG. 1, with a central axis 104. A set of reference axes are provided, indicating a propagation (e.g., signal propagation) direction 101, an azimuth direction 103, and an elevation direction 105. In other examples, the set of reference axes may represent a z-axis 101, an x-axis 103, and a y-axis 105. The piezoelectric element 102 is shown in FIG. 1 with the central axis 104 parallel with the propagation direction 101.

It will be noted that while the acoustic stack 100 is shown configured for a linear ultrasound probe and the propagation direction is described as parallel with the z-axis in FIG. 1, other examples may include a propagation direction that is angled relative to the z-axis, depending on a shape of a piezoelectric element array. For example, the ultrasound probe may be curvilinear or phased array, thus generating non-linear beams that are not parallel with the z-axis. Furthermore, while the examples shown and described herein are directed to ultrasound applications, the methods and systems described below may be applicable to a variety of sensor array types.

While a single piezoelectric element is shown in FIG. 1, the ultrasound probe may include a plurality of piezoelectric elements arranged in an array and individually coupled to an electrical energy source by wires. Each electrical circuit formed of one or more piezoelectric elements may be a transducer. In some examples, the transducer may include an array of piezoelectric elements which may arranged in a variety of patterns, or matrices, including one-dimensional (1D) linear, two-dimensional (2D) square, 2D annular, etc. Each transducer may be electrically insulated from adjacent transducers but may all be coupled to common layers positioned above and below the piezoelectric element, with respect to the propagation direction. The plurality of piezoelectric elements and accompanying layers may be enclosed by an outer housing of the ultrasound probe which may be, for example, a plastic case with a variety of geometries. For example, the outer housing may be a rectangular block, a cylinder, or a shape configured to fit into a user's hand comfortably. As such, components shown in FIG. 1 may be adapted to have geometries and dimensions suitable to fit within the outer housing of the ultrasound probe.

The piezoelectric element 102 may be a block formed of a material, such as lead zirconate titanate, that deforms and vibrates when a voltage is applied by, for example, a transmitter. In some examples, the piezoelectric element 102 may be a single crystal with crystallographic axes, such as PMN-PT (Pb(Mg_{1/3}Nb_{2/3})O₃—PbTiO). The vibration of the piezoelectric element 102 generates an ultrasonic signal formed of ultrasonic waves that are transmitted out of the ultrasound probe in a direction indicated by arrows 107, e.g., along the propagation direction 101. The piezoelectric element 102 may also receive ultrasonic waves, such as ultrasonic waves reflected from a target object, and convert the ultrasonic waves to a voltage. The voltage may be transmitted to a receiver of the ultrasound imaging system and processed into an image.

Electrodes 114 may be in direct contact with the piezoelectric element 102 to transmit the voltage via wires 115, the voltage converted from ultrasonic waves. The wires 115 may be connected to a circuit board (not shown) to which a plurality of wires from electrodes of the plurality of piezoelectric elements may be fixed. The circuit board may be coupled to a coaxial cable providing electronic communication between the ultrasound probe and the receiver. In one example, the circuit board may be one or more ASICs electrically coupled to the piezoelectric element 102 by an electrical interfacing structure. A configuration of the electrical interfacing structure may affect an overall size and efficiency of the ultrasound probe. Details of an electrical interfacing structure that maximizes an active area of the ultrasound probe relative to its overall area are provided further below with reference to FIGS. 3-13.

An acoustic matching layer 120 may be arranged above the piezoelectric element 102, with respect to the propagation direction 101, oriented perpendicular to the central axis 104. The acoustic matching layer 120 may be a material positioned between the piezoelectric element 102 and a target object to be imaged. By arranging the acoustic matching layer 120 in between, the ultrasonic waves may first pass through the acoustic matching layer 120, and emerge from the acoustic matching layer 120 in phase, thereby reducing a likelihood of reflection at the target object. The acoustic matching layer 120 may shorten a pulse length of the ultrasonic signal, thereby increasing an axial resolution of the signal.

A backing layer 126 may be arranged below the piezoelectric element 102, with respect to the propagation direction 101. In some examples, the backing layer 126 may be a block of material that extends along the azimuth direction 103 (and the elevation direction 105) so that each of the plurality of piezoelectric elements in the ultrasound probe are directly above the backing layer 126, with respect to the propagation direction 101. The backing layer 126 may be configured to absorb ultrasonic waves directed from the piezoelectric element 102 in a direction opposite of the direction indicated by arrows 107 and attenuate any stray ultrasonic waves deflected by the transducer and probe in directions other than directions useful for imaging, e.g., directions outside of a range of signal angles that may be transmitted and received by the ultrasound probe based on its specific size and frequency range. A bandwidth of the ultrasonic signal, as well as the axial resolution, may be increased by the backing layer 126.

In some examples, the backing layer 126 may be positioned under (e.g., with respect to the z-axis 101) at least one ASIC of the ultrasound probe. In such examples, the backing layer 126 may be formed from a continuous, e.g., undiced, material. Dicing of the backing layer 126 may be challenging due to a thickness of the backing layer 126, which may be greater than other layers of the acoustic stack 100. In other examples, however, the acoustic stack 100 may also include a dematching layer (not shown in FIG. 1) arranged directly below the piezoelectric element 102 and between the piezoelectric element 102 and the backing layer 126. The dematching layer may be a high acoustic impedance layer that reflects a majority of the ultrasonic signal received by the ultrasound probe out of a front of the ultrasound probe (e.g., along the propagation direction 101), allowing a reflected portion of the ultrasonic signal to be used for imaging.

An array of elements, each of the elements including the matching layer 120, the piezoelectric element 102, and the backing layer 126 of FIG. 1, may be coupled to one or more ASICs in an EAM for a transducer probe. As described above, the EAM may include an acoustic stack formed of the array of elements and an electrical interfacing structure that electrically couples the acoustic stack to the ASICs. In one example, e.g., a conventional example, the acoustic stack may be electrically coupled to the ASICs by a multilayer flex interconnect. The multilayer flex interconnect may be a single-or double-sided circuit board with flexible, conductive layers separated by flexible insulating layers. The layers may be interconnected by plated through-holes. An embodiment of a conventional EAM 200 relying on a multilayer flex interconnect 202 is depicted in FIG. 2.

An exploded view of the EAM 200 is shown in FIG. 2, where the EAM 200 may be formed by coupling an acoustic stack 204 to a first side 206 of the multilayer flex interconnect 202 and ASICs 208 to a second side 210 of the multilayer flex interconnect 202. The EAM 200 is shown from a cross-sectional profile view, e.g., along the x-z plane, where the acoustic stack 204 may be a 2D array of elements 212, each of the elements 212 including layers such as a matching layer, a piezoelectric material, and a backing layer as described above with reference to FIG. 1.

Each of the elements 212 may be aligned along the propagation direction 101 with one of a plurality of interconnect bumps 214 of the multilayer flex interconnect 202, allowing each of the elements 212 to be in face-sharing contact with one of the plurality of interconnect bumps 214 when the acoustic stack 204 is coupled to the first side 206 of the multilayer flex interconnect 202. The plurality of interconnect bumps 214 may be electrical contacts that provide electrical continuity between the first side 206 and the second side 210 of the multilayer flex interconnect 202. A pitch 216 of the elements 212 may therefore be similar or equal to a pitch 218 of the plurality of interconnect bumps 214.

The ASICs 208 also include bumps 220 (hereafter, ASIC bumps), which have a pitch 222 that is equal to the pitch 216 of the elements 212 and to the pitch 218 of the plurality of interconnect bumps 214. The ASIC bumps 220 may be in face-sharing contact with the plurality of interconnect bumps 214, e.g., each of the ASIC bumps 220 may be in face-sharing contact with one of the plurality of interconnect bumps 214, when the ASICs 208 are coupled to the second side 210 of the multilayer flex interconnect 202.

By aligning each of the elements 212 with one of the plurality of interconnect bumps 214 and one of the ASIC bumps 220 along the propagation direction 101, where each element/interconnect bump/ASIC bump combination forms a transducer, electrical continuity is enabled between the acoustic stack 204 and the ASICs 208. An active area 224 of the EAM 200 may be defined by an area occupied by the elements 212 along the x-y plane.

As a result of the alignment of the acoustic stack 204, the multilayer flex interconnect 202, and the ASICs 208, I/O connectors 226 of the multilayer flex interconnect 202 and corresponding I/O connectors 228 of the ASICs 208 may be located peripheral to the active area 224. For example, the I/O connectors 226, 228 may occupy overhead regions 230 of the EAM 200, which may be regions outside of and adjacent to the active area 224, along the x-y plane. The I/O connectors 226, 228 may provide electrical signals for power distribution, and digital communication to be transmitted to and from the EAM 200. By positioning the I/O connectors 226, 228 outside of the active area 224, an overall area 231 of the EAM 200, and therefore of a transducer probe incorporating the EAM 200, may be larger than the active area 224. For example, a ratio of the active area 224 to the overall area 231 may be less than one. The overhead regions 230 therefore represent areas of the EAM 200 that do not contribute to an imaging aperture of the transducer probe, which may constrain data acquisition capabilities of the EAM 200. In particular, for a transducer probe used to access tight spaces, such as for invasive applications, an active aperture of the transducer probe may not be optimized due to the overhead regions 230 occupied by the I/O connectors 226, 228.

Furthermore, fabricating the EAM such that the acoustic stack, the multilayer flex interconnect, and the ASICs are aligned may demand customized manufacturing of the EAM according to a specific type of transducer probe relying on the EAM. Such individualized product development may be time-consuming and costly. In addition, both TX and RX circuits, demanding high voltages and low voltages, respectively, may be incorporated into a single ASIC in the EAM which may inhibit performance optimization of the ASICs.

In one example, the issues described above may be at least partially addressed by manufacturing an EAM as a CSP, including an acoustic stack, one or more ASICs, and an interconnect with a fan-out architecture, e.g., a fan-out architecture interconnect. Hereafter, the EAM may be referred to as a CSP EAM. The fan-out architecture interconnect may enable the acoustic stack to be electrically coupled to the ASICs without demanding alignment of the acoustic stack elements with the ASIC bumps or peripheral positioning of I/O connectors relative to an active area of the CSP EAM. The active area may be maximized as a result, allowing an active aperture of a corresponding transducer probe to be increased. Increasing the active aperture may for example, enhance image quality when the transducer probe is used to acquire images. Additionally, the fan-out architecture interconnect may enable ASICs of a given configuration to be coupled to different acoustic stacks for a variety of transducer probe types, thereby precluding customized manufacturing of the CSP EAM. As well, a performance of the ASICs may be optimized by assigning TX and RX circuits to separate die, which may decrease an overall footprint of the ASICs within the CSP EAM and increase a manufacturing yield of the ASICs. Details and embodiments of CSP EAMs incorporating a fan-out architecture interconnect are depicted in FIGS. 3-13 and described further below.

Turning now to FIG. 3, components of a CSP EAM for a transducer probe are shown. For example, an acoustic stack 300, which may be similar to the acoustic stack 204 of FIG. 2, may be coupled to a fan-out architecture interconnect 302 which, in turn, may be coupled to one or more ASICs 304. A first example of a CSP EAM 400 formed by coupling the acoustic stack 300 to the fan-out architecture interconnect 302 is depicted in FIG. 4. The acoustic stack 300 may include elements 306 arranged in an array and coupled to an acoustic stack interposer 308, the acoustic stack interposer 308 configured to interface with the fan-out architecture interconnect 302.

For example, the acoustic stack interposer 308 may include interposer bumps 309 protruding from opposite sides of the acoustic stack interposer 308. The interposer bumps 309 may be aligned, along the z-axis 101, with the elements 306 of the acoustic stack 300, and may be fixedly coupled to a bottom surface (relative to the z-axis 101) of each of the elements 306. The acoustic stack interposer 308 may be fixedly coupled to the acoustic stack 300 by one or more of thermocompression, soldering, conductive epoxy, ultrasonic bonding, etc.

In one example, as shown in FIG. 3, the ASICs 304 may be enclosed within interconnect layers 310 of the fan-out architecture interconnect 302. The interconnect layers 310 may be electrically conductive routing layers extending across an entire width of the fan-out architecture interconnect 302 (the width defined along the x-axis 103) which are electrically interconnected by interconnect bumps 312. Further, the interconnect layers 310 may form a multi-layer interposer that is flex-based, rigid organic substrate-based, or silicon wafer-based. The interconnect bumps 312 may be arranged in the interconnect layers 310 such that an area occupied by the interconnect bumps 312 along the x-y plane decreases with each of the interconnect layers 310 along the propagation direction 101, away from the acoustic stack 300. For example, a first layer 310a (e.g., a top layer) of the interconnect layers 310 may have a largest area occupied by the interconnect bumps 312 while a second layer 310b (e.g., a bottom layer) of the interconnect layers 310 may have a smallest area occupied by the interconnect bumps 312. The areas occupied by the interconnect bumps 312 may decrease progressively between the first layer 310a and the second layer 310b.

Further, the interconnect bumps 312 of the first layer 310a of the interconnect layers 310 may protrude outwards, in a direction away from the second layer 310b, from the first layer 310a, along a top side of the fan-out architecture interconnect 302. The interconnect bumps 312 of the second layer 310b of the interconnect layers 310 may protrude both above (relative to the z-axis 101) and below the second layer 310b. The interconnect bumps 312 that protrude below the second layer 310b protrude outwards, in a direction away from the first layer 310a, along a bottom side of the fan-out architecture interconnect 302.

The interconnect bumps 312 of the second layer 310b of the interconnect layers 310 that protrude below the second layer 310b may be in direct contact with a first set of bumps 314 of the ASICs 304 (e.g., a first set of ASIC bumps 314). The contact between the interconnect bumps 312 of the second layer 310b and the first set of ASIC bumps 314, as well as the contact between interconnect bumps 312 of the first layer 310a and the interposer bumps 309 of the acoustic stack interposer 308, may provide electrical continuity between the acoustic stack 300 and the ASICs 304 when the acoustic stack is coupled to the fan-out architecture interconnect 302, as shown in FIG. 4.

However, in other examples, the interconnect bumps 312 of the second layer 310b and the first set of ASIC bumps 314 may not be in direct contact. Instead, the bumps may be electrically connected by routing traces. Furthermore, along internal layers of the interconnect layers 310, where the internal layers are layers of the interconnect layers 310 that do not have outward-facing bumps, such as a layer of the interconnect layers 310 between the first layer 310a and the second layer 310b, the bumps (e.g., internally located interconnect bumps 312) may or may not be of a same type as bumps that protrude outwards from the fan-out architecture interconnect 302. For example, the bumps 312 coupled to the layer between the first layer 310a and the second layer 310b may be different from the interconnect bumps protruding above the first layer 310a and/or from the interconnect bumps 312 of the second layer 310b that interface with the first set of ASIC bumps 314. The internally located interconnect bumps 312 may be electrically conductive pads that connect routing traces between the interconnect layers 310.

The fan-out architecture interconnect 302 may also include peripheral layers 316, which may be located around a periphery of the ASICs 304 along the x-y plane. The peripheral layers 316 therefore do not extend across the entire width of the fan-out architecture interconnect 302. Instead, the peripheral layers 316 have widths that extend across a portion of the width of the fan-out architecture interconnect 302. The peripheral layers 316 may also include routing layers electrically interconnected by electrical bumps, where the electrical bumps may be I/O bumps 318. At least a portion of the I/O bumps 318 may be in direct, e.g., face-sharing contact, with a second set of ASIC bumps 324 of the ASICs 304. Another portion of the I/O bumps 318 may be coupled to a bottom face (e.g., relative to the z-axis 101) of the second layer 310b of the interconnect layers 310.

A bottom layer 320 of the fan-out architecture interconnect 302 may extend across the entire width of the fan-out architecture interconnect 302 under (relative to the z-axis or propagation direction 101) the ASICs 304 and the peripheral layers 316. The bottom layer 320 may be electrically coupled to one of the peripheral layers 316 by the I/O bumps 318. The bottom layer 320 may further include bottommost bumps, or I/O connections 322 that protrude from a bottom face of the bottom layer 320, relative to the z-axis 101.

The I/O bumps 318 may maintain electrical continuity between the interconnect layers 310, the ASICs 304, the peripheral layers 316, and the bottom layer 320 of the fan-out architecture interconnect 302. Electrical signals may thereby be transmitted between the acoustic stack 300, the fan-out architecture interconnect 302, and I/O devices (not shown) connected to the I/O connections 322 of the fan-out architecture interconnect 302. The I/O devices may be other probe electronics, such as printed circuit boards (PCBs), as one example. As shown in FIG. 4, when the acoustic stack 300 is coupled to the fan-out architecture interconnect 302, the CSP EAM 400 may be a single structure incorporating the ASICs 304 enclosed within the fan-out architecture interconnect 302.

For example, the ASICs 304 may be surrounded by the interconnect layers 310, the peripheral layers 316, and the bottom layer 320 of the fan-out architecture interconnect 302 of the CSP EAM 400. The interconnect layers 310 may be arranged between the acoustic stack 300 and the ASICs 304 and the bottom layer 320 may be arranged between the ASICs 304 and the I/O devices electrically coupled to the I/O connections 322 of the bottom layer 320. As described above, a perimeter of the ASICs 304 may be surrounded by the peripheral layers 316. Electrical continuity between the acoustic stack 300 and the ASICs 304 is enabled by the acoustic stack interposer 308, the interconnect bumps 312 of the interconnect layers 310 (as well as the interconnect layers 310 therebetween), and the first set of ASIC bumps 324, while electrical continuity between the ASICs 304 and the I/O devices is enabled by the second set of ASIC bumps 324, the I/O bumps 318, and the I/O connections 322, as well as the peripheral layer 316 and the bottom layer 320 arranged therebetween, respectively.

In the CSP EAM 400, the interconnect bumps 312 of the first layer 310a may be in direct contact with the interposer bumps 309 of the acoustic stack interposer 308. The interposer bumps 309 of the acoustic stack interposer 308 may therefore have a pitch (e.g., a distance between centers of adjacent bumps) that is substantially equal to a pitch 402 of the elements of the acoustic stack 300. The interposer bumps 309 protruding from the bottom of the acoustic stack interposer 308 may be in direct contact (e.g., face-sharing contact) with the interconnect bumps 312 protruding outwards from the first layer 310a of the interconnect layers 310.

A pitch 402 of the elements 306 of the acoustic stack 300 may be different from a pitch 404 of the first set of ASIC bumps 314 of the ASICs 304. In one example, the pitch 402 of the elements 306 may be larger than the pitch 404 of the first set of ASIC bumps 314. A pitch of the interconnect bumps 312 may vary per layer of the interconnect layers 310 between a maximum pitch that is substantially equal to the pitch 402 of the elements 306 at the first layer 310a and a minimum pitch that is substantially equal to the pitch 404 of the first set of ASIC bumps 314 at the second layer 310b. A decrease in the pitch of the interconnect bumps 312 from the first layer 310a to the second layer 310b may correspond to the decrease in area of the interconnect bumps 312 with each descending layer, as described above, thereby allowing the acoustic stack 300 to be electrically connected to the ASIC 304 in spite of the difference in respective pitches.

Although the interconnect bumps 312 are depicted having a sequentially decreasing pitch according to layer, in other examples, the pitch of the interconnect bumps 312 may not decrease per layer in the sequential manner as shown. For example, with reference to the fan-out architecture interconnect 302 of FIGS. 3-4, a middle layer of the interconnect layers 310 (e.g., a layer in between the first layer 310a and the second layer 310b) may have interconnect bumps 312 with a pitch that is similar to the pitch 402 or the pitch 404, rather than a pitch therebetween. The pitches of the interconnect bumps 312 according to layer may therefore decrease (or increase) in a non-continuous manner.

In addition, individual electrical circuits corresponding to each of the elements 306 may be maintained in the fan-out architecture interconnect 302. By obviating a demand for alignment of the elements 306 with the first set of ASIC bumps 314, a footprint, e.g., a width of the ASIC 304 along the x-axis 103, may be decreased relative to the ASICS 208 of FIG. 2. As such, an active area 406 of the CSP EAM 400 may be similar, e.g., substantially equal, to an overall area 408 of the CSP EAM 400, increasing an active area to overall area ratio of the CSP EAM 400 relative to the EAM 200 of FIG. 2.

The decrease in footprint of the ASICs 304 allows the I/O connections 322 to be routed to an underside of the CSP EAM 400, rather than at peripheral locations relative to the active area 406. The fan-out architecture may therefore be provided in a CSP as a structure that may be readily fabricated to be usable with different acoustic stacks. For example, the first layer 310a of the interconnect layer 310 may be customized according to the pitch 402 of the elements 306 of the acoustic stack 300 but the pitches of all other layers of the fan-out architecture interconnect 302 may be independent of the acoustic stack configuration. The first layer 310a of the interconnect layer 310 may therefore be fabricated according to properties of an acoustic stack to which the fan-out architecture interconnect 302 is to be coupled. Bonding of the acoustic stack 300 to the fan-out architecture interconnect 302 to form the CSP EAM 400 may be achieved by methods including but not limited to thermocompression, soldering, conductive epoxy, ultrasonic bonding, etc.

The fan-out architecture interconnect may have two-sided connections (e.g., bumps) which provide electrical continuity between the acoustic stack and the ASICs. For a given type of ASIC die, the same ASIC die may be incorporated into different packages for different transducer probes, which may reduce development costs and time. Furthermore, a horizontal electrical routing provided by the fan-out architecture interconnect may allow for heterogeneous integration of the ASIC die, where different types of ASIC die may be packaged together into one CSP EAM. For example, a pitch of the interconnect bumps may not be uniform across the width of the CSP EAM. Instead, as indicated in FIG. 4, a first portion 410 of the interconnect bumps 312 may be electrically connected to a left ASIC of the ASICs 304 of FIG. 4, and be configured to match a pitch of the left ASIC. A second portion 412 of the interconnect bumps 312 may be electrically coupled to a right ASIC of the ASICs 304 of FIG. 4, and be configured to match a pitch of the right ASIC. The first portion 410 of the interconnect bumps 312 may therefore have different pitches than the second portion 412 of the interconnect bumps 312 for a given routing layer. The ASICs may thereby have different ASIC bump pitches and remain electrically coupled to the acoustic stack.

For example, one of the ASICs may be a TX circuit demanding approximately 180 nanometer-scale, high voltage technology while another of the ASICs may be a RX circuit demanding deep sub-micron-scale technology for lower voltage applications, where the TX circuit may be on a larger process than the RX circuit. Separating the TX and RX processes into separate die may lower costs as well as power consumption due to an ability to optimize electrical properties each of the die. Additionally, the separate die may be reused for different projects, thus enabling faster development of new projects.

It will be appreciated that the CSP EAM 400 illustrated in FIG. 4 is a non-limiting example of an EAM incorporating a fan-out architecture interconnect. Other examples may include variations in a number of interconnect bumps, I/O bumps, routing layers, ASIC bumps, etc., as well as different dimensions of the EAM components. In addition, a number of ASIC die coupled to the fan-out architecture interconnect may vary for a given configuration of the fan-out architecture interconnect.

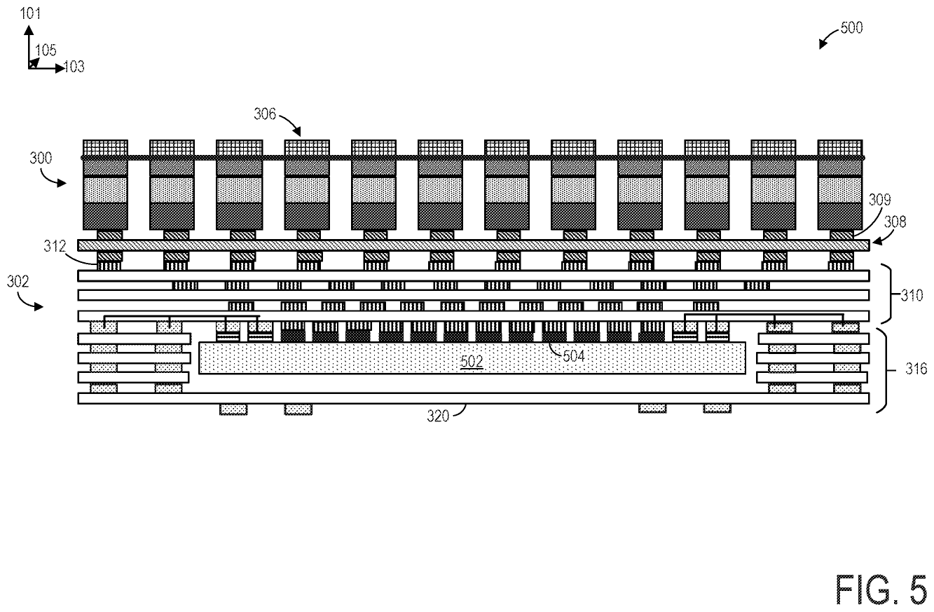

For example, a second example of a CSP EAM 500 is illustrated in FIG. 5. The CSP EAM 500 includes the acoustic stack 300 and the fan-out architecture interconnect 302 of FIGS. 3 and 4. Unlike the CSP EAM 400 of FIG. 4, however, the CSP EAM 500 includes a single ASIC 502 that may have an overall footprint similar to an overall footprint of the two ASICs 304 of the CSP EAM 400 of FIG. 4. A pitch of the ASIC bumps 504 is different from the pitch of the elements 306. The fan-out architecture interconnect 302 may therefore be readily adapted to different ASIC die and die quantities. For example, one, two, three, or more ASIC die may be incorporated in a given fan-out architecture interconnect configuration.

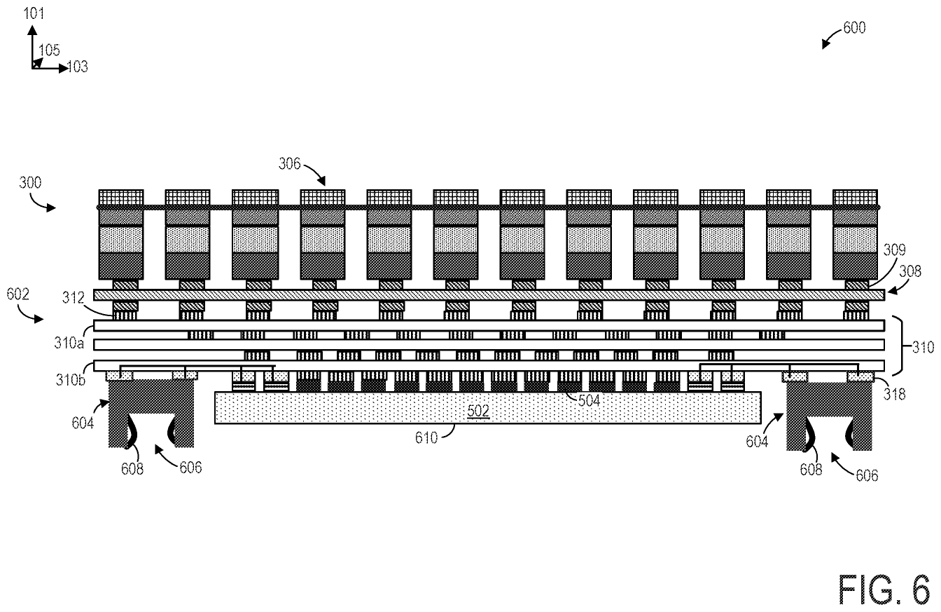

A third example of a CSP EAM 600 with a fan-out architecture interconnect 602 is shown in FIG. 6. The fan-out architecture interconnect 602 is coupled to the acoustic stack 300 via the acoustic stack interposer 308 of FIGS. 3-5. The ASIC 502 of FIG. 5 is also coupled to the fan-out architecture interconnect 602 of FIG. 6 in a similar manner. The fan-out architecture interconnect 602 further includes the interconnect layers 310 with the interconnect bumps 312, as shown in FIGS. 3-5, but does not include the peripheral layers 316 or the bottom layer 320 of the fan-out architecture interconnect 302 of FIGS. 3-5. In place of the peripheral layers, the fan-out architecture interconnect 602 has I/O connectors 604 coupled to the I/O bumps 318 of the second layer 310b of the interconnect layers, the I/O connectors positioned around a perimeter of the ASIC 502. For example, the I/O connectors 604 may be located peripheral to the ASIC 502.

The I/O connectors 604 may be connected to the I/O bumps 318 of the second layer 310b of the interconnect layers 310 and may be structures housing electrical components to provide electrical continuity between the ASIC 502 and I/O devices (not shown) connected to the I/O connectors 604. The I/O connectors 604 may include receiving ports 606 for receiving connectors of the I/O devices as well as electrical contacts 608 arranged in the receiving ports 606. The I/O connectors 604 may extend along the z-axis 101 below a bottom face 610 of the ASIC 502, on either side of the ASIC 502. The ASIC 502 is not enclosed within the fan-out architecture interconnect 602.

A configuration of the CSP EAM 600 enables in coupling of I/O devices to an underside of the CSP EAM 600, rather than in overhead regions, as shown in FIG. 2. Similar to the CSP EAM 500 of FIG. 5, an active area of the CSP EAM 600 may be maximized while providing flexibility with respect to ASIC incorporation. A pitch of the elements 306 is not tied to, e.g., not dependent on, a pitch of the ASIC bumps 504. The use of the I/O connectors 604 enables I/O devices with corresponding connectors to be readily coupled to the CSP EAM 600.

In one example, the configuration of the CSP EAM 600 may allow the CSP EAM 600 to be readily exchanged for another EAM. For example, the CSP EAM 600 is not permanently bonded to an I/O device coupled thereto via the I/O connectors 604. In an event that the CSP EAM 600 becomes degraded, the non-permanent arrangement of the I/O connectors 604 along the underside of the CSP EAM 600 may allow the CSP EAM 600 to be removed and replaced by a new EAM and the I/O device may remain unaltered. Furthermore, the CSP EAM 600 may provide assembly reworkability advantages. As an example, bonding of one or more EAMs concurrently to the I/O device may be undesirable due to a demand for discarding an entire corresponding assembly if a single EAM isn't bonded suitably. A flexible connectivity provided by the CSP EAM 600 therefore decreases manufacturing losses.

A third example of a CSP EAM 700 is illustrated in 700, the CSP EAM 700 also including the acoustic stack 300 and the ASIC 502, as well as a fan-out architecture interconnect 702. The fan-out architecture interconnect 702 includes the interconnect layers 310 but does not include the peripheral layers 316 of the CSP EAM 500 of FIG. 5, or the I/O connectors 604 of the CSP EAM 600 of FIG. 6. Instead, I/O devices may be electrically connected to the I/O bumps 318 of the second layer 310b of the interconnect layers 310. For example, the I/O bumps 318 of the second layer 310b may be gold bumps, copper pillars, or other analogous types of electrical contacts protruding from the bottom face of the second layer 310b. Electrical connections of I/O devices may thereby be bonded to the I/O bumps 318 along a bottom side of the CSP EAM 700 while an active area of the CSP EAM 700 is maximized. The CSP EAM 700 may be associated with lower costs compared to, for example, the CSP EAMs of FIGS. 4 and 5.

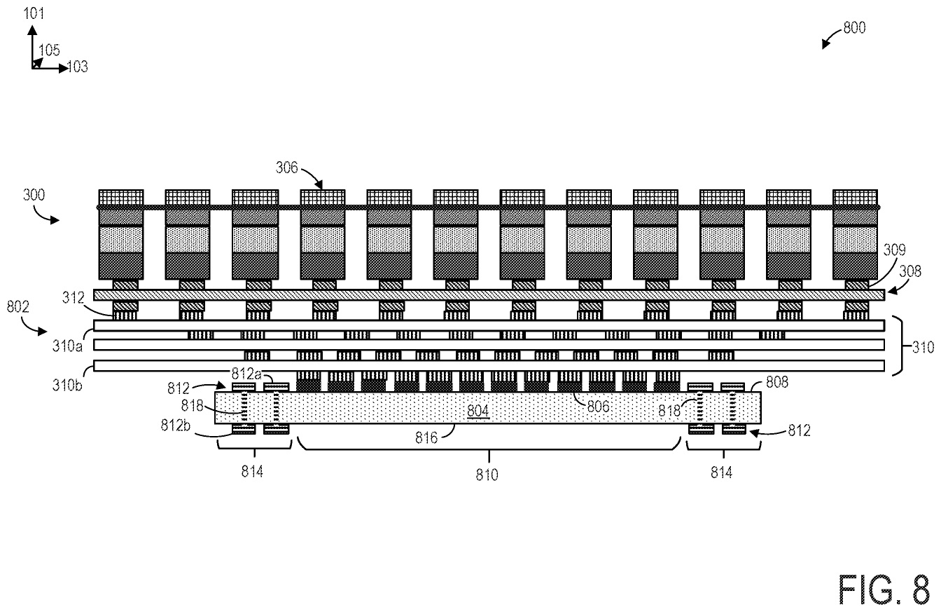

A fourth example of a CSP EAM 800 is shown in FIG. 8, which includes the acoustic stack 300 and a fan-out architecture interconnect 802 having the interconnect layers 310. The second layer 310b of the interconnect layers 310, however, does not include I/O bumps. The fan-out architecture interconnect 802 is electrically coupled to an ASIC 804 at an interface between the interconnect bumps 312 at the bottom face of the second layer 310b of the interconnect layers 310 and a first set of ASIC bumps 806 at an upper face 808 of the ASIC 804. The first set of ASIC bumps 806 may extend along a central region 810 of the ASIC 804.

The ASIC 804 further includes a second set of ASIC bumps 812, arranged at peripheral regions 814 of the ASIC 804 located on either side of the central region 810 of the ASIC 804. The second set of ASIC bumps 812 include upper bumps 812a, positioned at the upper face 808 of the ASIC 804, and bottom bumps 812b, positioned at a bottom face 816 of the ASIC 804. Each of the upper bumps 812a may be aligned with one of the bottom bumps 812b along the z-axis 101 and electrically coupled to one another by through-silicon vias (TSVs) 818 that pass through a thickness of the ASIC 804.

The upper bumps 812a may be electrically continuous with the first set of ASIC bumps 806, which are, in turn, electrically continuous with the bottom bumps 812b of the second set of ASIC bumps 812 due to the TSVs 818. I/O devices (not shown) may be connected to the bottom bumps 812b. The CSP EAM 800 of FIG. 8 may therefore provide an alternate electrical configuration for electrically coupling I/O devices to the CSP EAM 800 at the bottom face 816 of the ASIC 804 while maximizing an active area of the CSP EAM 800. An alignment of electrical connectors of the I/O devices may or may not be aligned with the bottom bumps 812b.

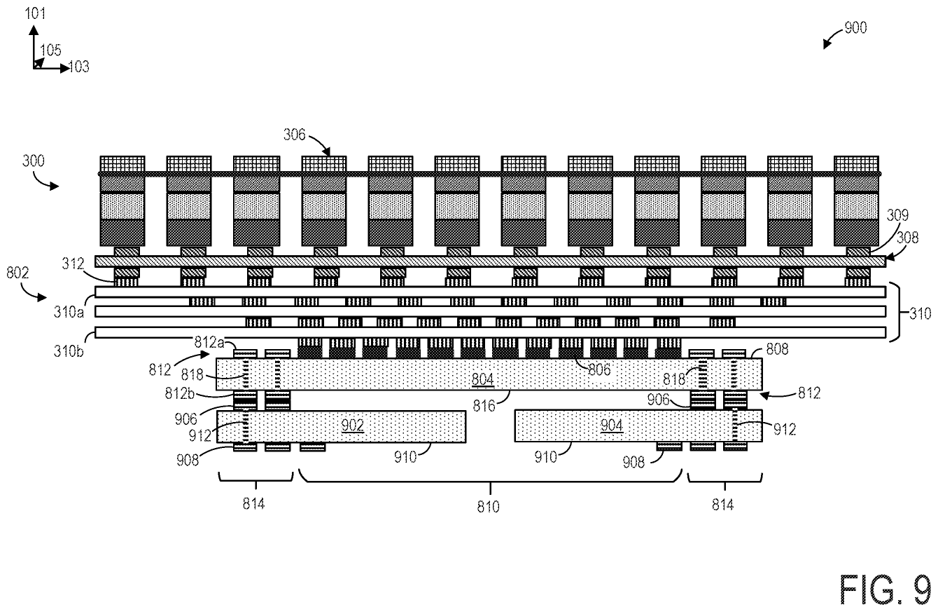

By utilizing TSVs to provide an electrical bridge through a thickness of an ASIC, a CSP EAM may be adapted with stacked ASICs, which may also incorporate heterogeneous ASICs. For example, a fifth example of a CSP EAM 900 is depicted in FIG. 9. The CSP EAM 900 includes components of the CSP EAM 900 of FIG. 8, such as the acoustic stack 300, the fan-out architecture interconnect 802, and the ASIC 804. A first additional ASIC 902 may be coupled to the second set of ASIC bumps 812 at one of the peripheral regions 814 of the ASIC 804 and a second additional ASIC 904 may be coupled to the second set of ASIC bumps 812 at the other of the peripheral regions 814 of the ASIC 804. The first and second additional ASICs 902, 904 may be stacked under (e.g., relative to the z-axis) the ASIC 804, aligned with and spaced away from one another along the x-y plane.

In one example, the ASIC 804 may be a combined TX/RX die, allowing ASIC die of different types to be coupled thereto. In another example, the ASIC 804 may be replaced with more than one ASIC of different die types, such as an individual TX die and an individual RX die. The first and second additional ASICs 902, 904 may be different types of die and may be coupled to the ASIC 804 accordingly. As one example, the first additional ASIC 902 may be a processor chip and the second additional ASIC 904 may be a memory chip.

The first and second additional ASICs 902, 904 may be electrically coupled to the bottom bumps 812b of the second set of ASIC bumps 812 of the ASIC 804. For example, the additional ASICs may each have top bumps 906 that interface with the bottom bumps 812b of the ASIC 804. The additional ASICs may further include lower bumps 908 protruding from their respective bottom faces 910. The lower bumps 908 may be electrically continuous with the lower bumps 908 of the first and second additional ASICs 902, 904 by way of TSVs 912 extending through thicknesses of the additional ASICs. I/O devices may be electrically connected to the CSP EAM 900 at the lower bumps 908 of the first and second additional ASICs 902, 904.

By incorporating ASICs with TSVs, an active area of a CSP EAM may be maintained large relative to its overall area while allowing more than one ASIC to be coupled to a fan-out architecture interconnect. The ASICs may be stacked along a propagation direction of the CSP EAM such that addition of ASICs does not increase an overall area of the CSP EAM. A footprint of the ASICs does not adversely affect the active area while ASICs used for different applications may be incorporated into a single EAM.

The types and quantities of ASIC die described above for incorporation into the CSP EAM 900 are non-limiting examples and may vary according to application. Similarly, aspects of the CSP EAMs of FIGS. 4-9, such as number, dimensions, and type of ASIC die, number of elements, number of fan-out architecture interconnect components, etc., may be varied in other examples without departing from a scope of the present disclosure.

In some examples, a large element array, e.g., of dimensions larger than a single EAM, may be desirable for a transducer probe. For EAMs with peripheral I/O connections, such as the EAM 200 of FIG. 2, a size of the array may be constrained to a single EAM due to a presence of the overhead regions surrounding the active area of the EAM. When the I/O connections are routed under an acoustic stack of the EAM, however, as depicted by the CSP EAMs illustrated in FIGS. 4-9, the EAMs may be tiled e.g., arranged adjacent and contiguous with one another along a common plane, to form a larger, continuous array of elements. The EAMs may be tiled along one or more dimensions, such as along an elevation direction of the transducer probe and/or an azimuth direction. A modular array is thereby provided where dimensions of the modular array may be selectable by adjusting a number of EAMs forming the array. It will be appreciated that the tiling of the EAMs may be analogously applied to any other type of sensor array demanding tiling of modules in any direction along a plane perpendicular to a direction of signal propagation. A first example of a continuous array 1000 formed of tiled EAMs is shown in FIG. 10. Each of the EAMs of the continuous array 1000 may be configured as the CSP EAM 400 of FIG. 4.

The continuous array 1000 includes a first EAM 1002, a second EAM 1004, and a third EAM 1006, although various other quantities of EAMs may be included, in other examples. As described above, with reference to FIG. 4, each of the EAMs has a fan-out architecture interconnect coupling an acoustic stack to ASICs, with I/O connections routed to a bottom of the EAMs. The EAMs may be aligned with one another along the x-y plane. Further, the EAMs arranged such that a space, e.g., a distance, between adjacent EAMs may be similar to a space between elements 1008 of the EAMs.

For example, an interelement kerf width 1010, e.g., a distance between adjacent elements 1008, may be similar to an intermodule kerf width 1012, e.g., a distance between adjacent elements 1008 of adjacent EAMs. In one example, the interelement kerf width 1010 may be substantially equal to the intermodule kerf width 1012. By maintaining the intermodule kerf width 1012 similar to the interelement kerf width 1010, a resulting array of the elements may be continuous across an active area 1014 of a corresponding transducer probe. In other words, the active area 1014 is not interrupted by large gaps between adjacent EAMs, as may otherwise occur if EAMs with I/O connections occupying overhead regions of each EAM are tiled to form a larger array.

Positions of the EAMs may be supported and maintained by coupling the EAMs to a common, unifying PCB 1018, as indicated by arrows 1020. For example, the EAMs may be attached to the unifying PCB 1018 via techniques such as thermocompression bonding, soldering, conductive epoxy, ultrasonic bonding, etc. The unifying PCB 1018 may extend across a width of the active area 1014 of the continuous array 1000 and may couple to bottom sides (e.g., relative to the z-axis 101) of the EAMs from which I/O connections 1022 protrude. The I/O connections 1022 may interface with bumps 1024 along an upper face 1026 of the unifying PCB 1018. The unifying PCB 1018 may include other, optional PCB components 1028 for processing, memory, etc., arranged along a lower face 1030 of the unifying PCB 1018. The lower face 1030 of the unifying PCB 1018 may also include a connector 1032 for electrically coupling the unifying PCB to another PCB.

Formation of a continuous array by tiling EAMs to have intermodule kerf widths that are similar to interelement kerf widths of the EAMs may therefore be enabled by configuring the EAMs with a fan-out architecture interconnect. The continuous array may be formed by any of the EAMs of FIGS. 4-9 due to a common positioning of I/O connections and/or connectors under the EAMs. In some examples, the continuous array may be homogeneous, e.g., formed from a common configuration of the EAMs, or the continuous array may be heterogeneous, e.g., formed from combinations of different EAM configurations. For example, the continuous array may include only the CSP EAM 500 of

FIG. 5. In another example, the continuous array may include the CSP EAM 400 of FIG. 4, the CSP EAM 600 of FIG. 6, and/or the CSP EAM 800 of FIG. 8. Numerous combinations and quantities of the EAMs incorporated into the continuous array are possible.

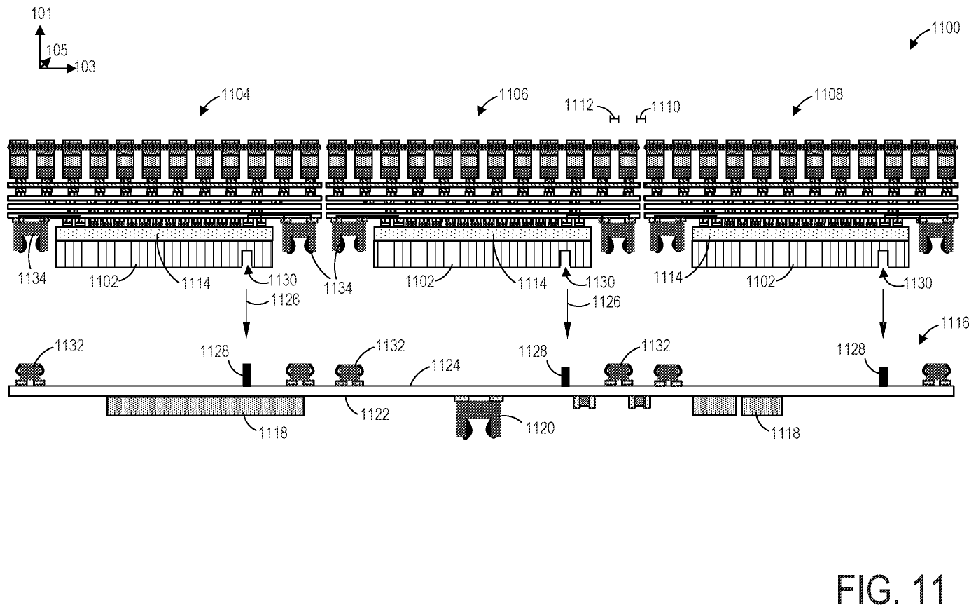

In some instances, adapting a continuous array with heat management capabilities may be desirable. For example, ASICs included in a transducer probe may dissipate power by releasing heat, which may degrade the transducer probe components if transmitted thereto. To alleviate thermally-induced degradation, a transducer probe may be adapted with thermal management devices, such as heat sinks, to route heat away from a patient contact surface of the transducer probe and towards other thermal management components, such as heat spreaders. A second example of a continuous array 1100 is illustrated in FIG. 11, the continuous array 1100 including thermal substrates 1102 coupled to each of a first EAM 1104, a second EAM 1106, and a third EAM 1108. Each of the EAMs are configured similar to the CSP EAM 600 of FIG. 6. The thermal substrates 1102 may be heat sinks that conduct heat away from ASICs 1114 or heat storage elements of the continuous array 1100, and/or absorb and trap thermal energy. When the thermal substrates 1102 are configured to conduct heat away from the ASICs 1114, the thermal substrates 1102 may be formed of high thermal conductivity metals, such as copper, aluminum, etc., as well as non-metals such as graphite, aluminum nitride, etc. When the thermal substrates 1102 are configured to store heat, the thermal substrates may be formed of high latent heat materials, such as phase change materials including paraffin wax.

As described above, intermodule kerfs 1110 of the continuous array 1100 may be similar to interelement kerfs 1112 thereof. The thermal substrates 1102 may be coupled to a bottom face of the ASICs 1114 of the EAMs and may protrude from a bottom side of the continuous array 1100. The continuous array 1100 may be coupled to a unifying PCB 1116, as described above with reference to FIG. 10. The unifying PCB 1116 may include optional PCB components 1118 and a connector 1120 for coupling the unifying PCB to another PCB along a bottom face 1122 of the unifying PCB 1116.

A top face 1124 of the unifying PCB 1116 may include alignment structures 1128 for guiding alignment, as indicated by arrows 1126, of the continuous array 1100 with the unifying PCB 1116 when the continuous array 1100 is coupled to the unifying PCB 1116. The alignment structures 1128 may be inserted into corresponding receiving slots 1130 of the thermal substrates 1102. The top face 1124 of the unifying PCB 1116 may also include electrical pins 1132 configured to be inserted into I/O connectors 1134 of the EAMs. When the unifying PCB 1116 is connected to the continuous array 1100 via engagement of the electrical pins 1132 with the I/O connectors 1134 and the alignment structures 1128 with the receiving slots 1130, the thermal substrates 1102 may be compressed between the EAMs and the unifying PCB 1116, allowing heat transfer from the ASICs 1114 to the unifying PCB 1116 and from the unifying PCB 1116 to other thermal management components such as heat spreaders (not shown).

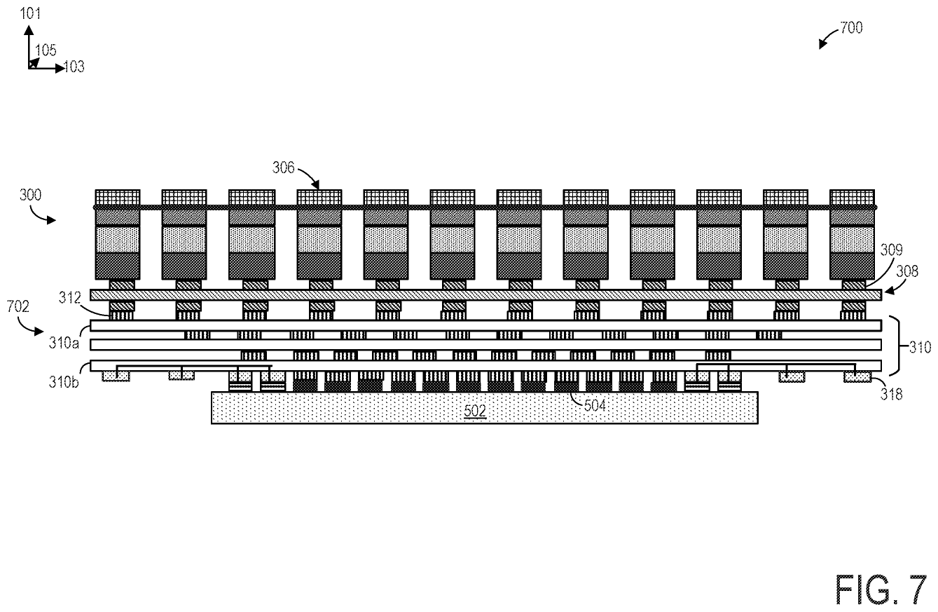

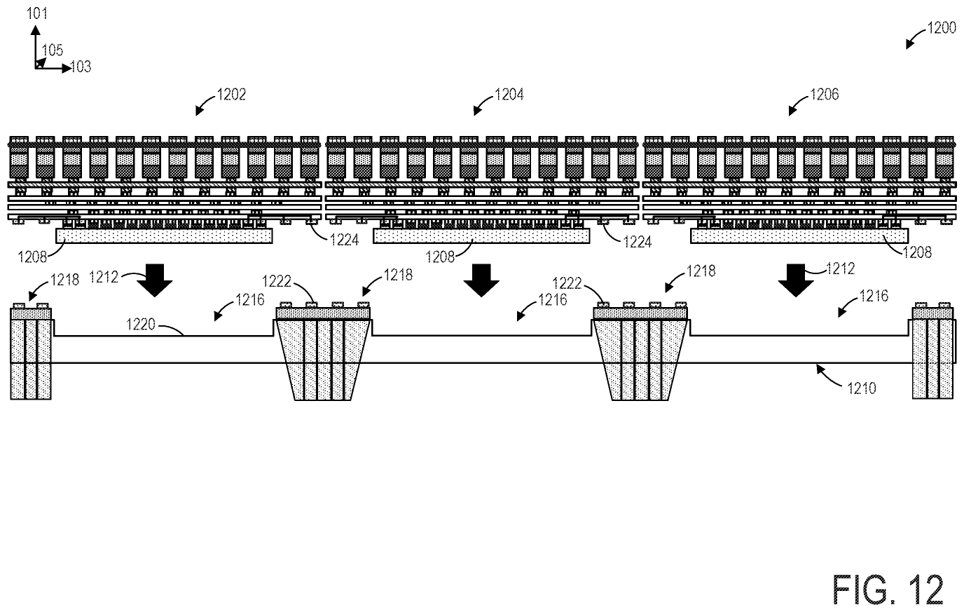

Alternatively, thermal management may be provided by a single structure coupled to each of the EAMs of a transducer probe, as shown in FIG. 12. A third example of a continuous array 1200 is depicted in FIG. 12, the continuous array 1200 including a first EAM 1202, a second EAM 1204, and a third EAM 1206. The EAMs are configured as the CSP EAMs 700 of FIG. 7, the EAMs including ASICs 1208.

The continuous array 1200 may be coupled to a common, unifying thermal substrate 1210, as indicated by arrows 1212. For example, the unifying thermal substrate 1210 may be attached to the continuous array 1200 by any of the techniques described previously, allowing the unifying thermal substrate to absorb or conduct heat from the continuous array 1200 as a heat sink. Further, the unifying thermal substrate 1210 may be formed of any of the materials described above, with respect to the thermal substrates 1102 of FIG. 11. The unifying thermal substrate 1210 may include recesses 1216 for receiving the ASICs 1208 when the continuous array 1200 is coupled to the unifying thermal substrate 1210. For example, the recesses 1216 may have dimensions along the x-axis 103, the y-axis 105, and the z-axis 101 that are similar to corresponding dimensions of the ASIC 1208. The ASICs 1208 may therefore be nested in the recesses 1216 of the unifying thermal substrate 1210 which may also guide alignment of the EAMs with the unifying thermal substrate 1210.

The unifying thermal substrate 1210 may also include electronic islands 1218 located between the recesses 1216 which may protrude along the z-axis 101, from an upper face 1220 of the unifying thermal substrate 1210. As such, the electronic islands 1218 may be spaced apart by a footprint of each of the ASICs 1208. The electronic islands 1218 may include bumps 1222, as well as other electronic structures and components, where the bumps 1222 may be configured to interface with I/O connections 1224 of the EAMs. Electrical continuity through the unifying thermal substrate 1210 may be provided by the electronic islands 1218.

Power dissipation at ASICs of a transducer probe may thereby be thermally managed by adapting the transducer probe with one or more heat sinks. The heat sink(s) may be individual structures directly coupled to the ASICs, as shown in FIG. 11, or may be a single structure interfacing with each EAM of the transducer probe, as shown in FIG. 12. In some examples, the transducer probe may include heat sink as both individual structures coupled to the ASICs and as a single structure commonly coupled to all the EAMs of the transducer probe.

While routing I/O connections and connectors to an underside of an EAM may be desirable for maximizing an active area or active aperture of a transducer probe, there may be instances where maintaining the I/O connections or connectors in peripheral, overhead regions may be demanded. Although the ratio of the active area to overall area of the transducer probe may be constrained to be less than one, adapting the EAM as a CSP with a fan-out architecture interconnect may still offer benefits with respect to cost and power optimization.

For example, an exemplary EAM 1300 is depicted in FIG. 13 with an acoustic stack 1302 coupled to an ASIC 1304 by a fan-out architecture interconnect 1306. The EAM 1300 includes I/O connectors 1308 arranged at an upper surface 1310 of a topmost layer 1312 of the fan-out architecture interconnect 1306. The I/O connectors 1308 may be positioned in overhead regions 1314, around an active area 1316 of the EAM 1300.

Although an overall area (e.g., a sum of the active area 1316 and the overhead regions 1314) of the EAM 1300 is larger than the active area 1316, by utilizing the fan-out architecture interconnect 1306 rather than a multilayer flex interconnect, such as the multilayer flex interconnect 202 of FIG. 2, a pitch 1318 of ASIC 1304 (e.g., of bumps 1320 of the ASIC 1304) may be independent of a pitch 1322 of the acoustic stack 1302 (e.g., of elements 1324 of the acoustic stack 1302). The pitch 1318 of the ASIC 1304 may be smaller than the pitch 1322 of the acoustic stack 1302, allowing a footprint, e.g., a size, of the ASIC 1304 to be reduced. By enabling incorporation of a smaller ASIC, a cost of the EAM may be decreased. Furthermore, in other examples, more than one ASIC die may be coupled to the fan-out architecture interconnect 1306, which may include ASIC die of different types.

In this way, a quality of data acquired by a transducer probe may be increased. An acoustic stack of the transducer probe may be electrically coupled to one or more ASICs by an interconnect (e.g., interposer) with a fan-out architecture, where the acoustic stack, the ASICs, and the interconnect may be packaged into a CSP EAM. The fan-out architecture of the interconnect may allow I/O connections to be located at a bottom side of the CSP EAM, thereby precluding occupation of the I/O connections in overhead regions. An active aperture of the CSP EAM may be maximized, which may increase a resolution, penetration, signal-to-noise ratio, etc., of the transducer probe, particularly when a footprint of the transducer probe is constrained due to application. The fan-out architecture interconnect may include bumps that transition in pitch between a pitch of the acoustic stack and a pitch of the ASICs. By removing a demand for a common pitch between the acoustic stack and the ASICs, a size of the ASICs may be decreased. For example, the pitch of the ASICs may be reduced relative to the acoustic stack pitch by up to 40% via the interconnect. As a cost of the ASICs may represent a significant portion of an EAM cost, e.g., as much as a third of an overall cost of an EAM, the decrease in size of the ASICs may result in a decrease in the overall cost of the EAM that exceeds any increase in cost of the fan-out architecture interposer relative to a conventional interconnect.

Furthermore, manufacturing of transducer probes may be more efficient and cost-effective by utilizing the fan-out architecture interconnect. As an example, a line of probes may be manufactured using a common ASIC, e.g., an ASIC of a given pitch and dimensions. The ASIC may be coupled to the interconnect with varying transitions in pitch according to a pitch of an acoustic array to which the ASIC is to be coupled. For example, the ASIC may be used in a transducer probe configured for high frequency operation. A corresponding high frequency acoustic stack may have a pitch of 50-200 μm, as an example, and the ASIC may be electrically coupled to the high frequency acoustic stack by a fan-out architecture interposer with a suitable transition in pitch.

The same ASIC may be used in a transducer probe configured for low frequency operation. A corresponding low frequency acoustic stack may have a pitch of several hundreds of microns, for example, and the ASIC may be electrically coupled thereto by a fan-out architecture interconnect with a suitable transition in pitch. The fan-out architecture may be selectively fabricated according to target pitch transitions to allow the same ASIC type to be used in a variety of probe types. Customized fabrication of the fan-out architecture may be less costly and faster than customized fabrication of ASICs. Time-consuming, end-use specific manufacturing of ASICs is thereby obviated.

For example, as shown in FIGS. 14A-14B, an ASIC of a given size or footprint (e.g., a given length and width along the x-y plane) may be coupled to acoustic stacks with varying dimensions, such as varying active apertures or areas. As shown in FIG. 14A, a first CSP EAM 1400 may have a first acoustic stack 1402 coupled to a first ASIC 1404 by a first fan-out architecture interconnect 1406. The first ASIC 1404 may have a footprint 1408 that is smaller than an active area 1410 of the first CSP EAM 1400.

A second CSP EAM 1450 is depicted in FIG. 14B. The second CSP EAM 1450 may have a second acoustic stack 1452 coupled to a second ASIC 1454 by a second fan-out architecture interconnect 1456. The second ASIC 1454 may be of a same type and size as the first ASIC 1404. A footprint 1458 of the second ASIC 1454 may be smaller than an active area 1460 of the second acoustic stack 1452 but the active area 1460 of the second acoustic stack 1452 is smaller than the active area 1410 of the first acoustic stack 1402. Different acoustic stacks may therefore be coupled to a common ASIC (e.g., a given type of ASIC with a given footprint) by modifying a geometry of the fan-out architecture connect, obviating a demand for altering the ASIC to accommodate a configuration of the acoustic stack. The acoustic stack footprint may vary between a ratio of the acoustic stack active area to the ASIC area of one to ratios of greater than one. Thus, in some examples, a largest footprint of the ASIC may be substantially equal to the active area of the acoustic stack. Conversely, a smallest footprint of the acoustic stack may be substantially equal to the footprint of the ASIC (or ASICS).

A configuration (e.g., a number of layers, a quantity of interconnect bumps, a pitch of the interconnect bumps per layer, etc.) of the fan-out architecture interconnect may be determined based on the relative footprints of the acoustic stack (e.g., the active area) and the ASIC (or ASICs). For example, the fan-out architecture may be fabricated to enable transitioning between the acoustic stack pitch and a pitch of the ASIC bumps, with a suitable number of layers and a suitable change in bump pitch per layer of the fan-out architecture interconnect.

As an example, a line of transducer probes, such as ultrasound probes, may be manufactured in a cost-efficient manner by configuring each of the probes with an ASIC of a common type and footprint. For example, as shown in FIG. 17, a line of ultrasound probes 1700 may include a first probe 1702, a second probe 1704, and a third probe 1706, the probes being of different probe types. Each of the probes includes an ASIC 1708 of a given type and footprint 1710.

The probes may have different acoustic stacks 1703 with different footprints, or active apertures. For example, the first probe 1702 has a first active aperture 1712 that is larger than the footprint 1710 of the ASIC 1708 and also larger than a second active aperture 1714 of the second probe 1704. The second active aperture 1714 is substantially equal to the footprint 1710 of the ASIC 1708. The third probe 1706 has a third active aperture 1716 that is larger than both the first aperture 1712 of the first probe and the second active aperture 1714 of the second probe 1704. Each of the probes may include a fan-out architecture interconnect 1718 with a footprint (e.g., area along the x-y plane) that corresponds to the active aperture of the respective probe.

The probes of the line of ultrasound probes 1700 may therefore share ASICs of a common size and type but incorporate different fan-out architecture interconnects and different acoustic stacks. A given type and size of ASIC may thereby be used in different types of transducer (e.g., ultrasound) probes. Furthermore, for a line of ultrasound probes manufactured to each incorporate a common ASIC, the common ASIC may have a footprint, e.g., area, that is no larger than an ultrasound probe having a smallest active aperture of the line of ultrasound probes. Thus, a size of the common ASIC used may be constrained by a size of a smallest ultrasound probe to be manufactured and/or the size of the smallest ultrasound probe manufactured may be constrained by the size of the common ASIC.

In addition, use of the fan-out architecture interconnect may enable different types of ASICs to be used in a multi-frequency transducer probe. For example, different EAMs, each having different acoustic stack pitches, may be incorporated into the multi-frequency transducer probe. The multi-frequency transducer probe may include a high frequency EAM and a low frequency EAM, as an example. By separating the acoustic stack pitches from the ASIC pitches, active apertures of the multi-frequency transducer probe may be maximized.

In yet another example, a fan-out architecture interconnect may enable a curvilinear probe to have a maximized active aperture and provide electrical continuity between an acoustic stack and at least one ASIC. For example, a curvilinear probe is illustrated in FIG. 18, which, in one example, may be a convex module 1800. The convex module 1800 may include an acoustic stack 1802 with layers of acoustic materials such as piezoelectric elements, matching layers, dematching layers, backing layers, etc., similar to the acoustic stacks shown previously. The acoustic stack may be curved, e.g., along the plane perpendicular to the propagation direction 101.

A fan-out architecture interconnect 1804 may be curved to match a curvature of the acoustic stack 1802, the fan-out architecture interconnect 1804 having a similar configuration to any of the previous examples and coupled to the acoustic stack 1802 in a manner as described above. The convex module 1800 may have an active area 1806 that is maximized relative to an overall footprint of the convex module 1800. For example, by coupling the acoustic stack 1802 to the fan-out architecture interconnect 1804, a presence of overhead regions is precluded.

An ASIC 1808 may be attached to the fan-out architecture interconnect 1804 opposite of the acoustic stack. In one example, a thickness, as measured along the propagation direction 101, of the ASIC 1808 may be reduced relative to ASICs coupled to planar modules, e.g., as shown in FIGS. 3-14B and 17. By reducing the thickness of the ASIC 1808, a material of the ASIC 1808 may be more flexible, allowing the ASIC 1808 to be curved to match a curvature of the fan-out architecture interconnect 1804.

An example of a method 1500 for assembling a CSP EAM for a transducer probe is shown in FIG. 15. The CSP EAM may be any of the CSP EAMs depicted in FIGS. 4-9, 13, and 18. The method may be executed at a manufacturing facility at which individual components of the CSP EAM may or may not be fabricated by one or more of manual and automated processes.

At 1502, the method includes fabricating a fan-out architecture interconnect according to target pitches of an acoustic stack and at least one ASIC of the CSP EAM. For example, a top layer of the fan-out architecture interconnect configured to interface with the acoustic stack may have a pitch that matches a pitch of the acoustic stack. A bottom layer of the fan-out architecture interconnect configured to interface with the ASIC may have a pitch that matches a pitch of the ASIC. Intermediate layers of the fan-out architecture may have pitches that decrease sequentially in a direction from the top layer to the bottom layer.

At 1504 of the method, the ASIC may be coupled to the bottom layer of the fan-out architecture interconnect by various techniques, including thermocompression, soldering, conductive epoxy, ultrasonic bonding, amongst others. At 1506, the acoustic stack may be coupled to the top layer of the fan-out architecture interconnect. For example, the acoustic stack may include an interposer with electrical bumps aligned with electrical bumps of the top layer of the fan-out architecture interconnect. The acoustic stack may be attached to the fan-out architecture interconnect by any of the aforementioned bonding techniques.

In other examples, 1504 and 1506 of the method may be reversed. For example, the acoustic stack may be coupled to the fan-out architecture interconnect before the ASIC is coupled to the fan-out architecture interconnect. In yet other examples, the acoustic stack and the ASIC may be coupled to the fan-out architecture interconnect concurrently. After coupling of the acoustic stack and the ASIC to the fan-out architecture (according to any of order of coupling described above) the CSP EAM may then be incorporated into the transducer probe individually or may be tiled with other EAMs to form a continuous array.

A method 1600 for manufacturing a line of transducer probes incorporating a common ASIC is shown in FIG. 16. For example, the transducer probes may vary in footprint and may have different active areas or apertures. However, each of the transducer probes may include an ASIC of a given area or footprint which may also be of a given type. Each of the transducer probes may be configured with one or more of the CSP EAMs of FIGS. 4-9, and 13, or the continuous arrays of FIGS. 10-12, where the continuous arrays may incorporate any combination of the CSP EAMs.

At 1602, the method includes obtaining ASICs with a given footprint (e.g., area along a plane perpendicular to a propagation direction of the transducer probes). Obtaining the ASICs may include fabricating the ASICs, such as by a wafer-scale manufacturing process, or acquiring the ASICs from a manufacturer. The ASICs may therefore be of common dimensions and of a common type.

At 1604, the method includes attaching different fan-out architecture interconnects to each of the ASICs to form ASIC assemblies. The fan-out architecture interconnects may have different configurations and dimensions. For example, the fan-out architecture interconnects may vary in number of interconnect layers, number of interconnect bumps, interconnect bump pitches per interconnect layer, etc., according to a configuration of a target acoustic stack to be coupled to the respective ASIC assembly. A pitch of the interconnect bumps at a bottom layer of a respective fan-out architecture interconnect may match a bump pitch of an ASIC to which the respective fan-out architecture interconnect is coupled to. A minimum size or footprint of the fan-out architecture interconnects may correspond to the footprint of the ASICs. Each of the fan-out architecture interconnects may be selected based on a configuration of an acoustic stack to be coupled thereto.

Acoustic stacks are coupled to each of the ASIC assemblies at 1606. The acoustic stacks may vary in active area size and may have pitches that match a pitch of the interconnect bumps at a top layer of a fan-out architecture interconnect to which a respective acoustic stack is coupled. The acoustic stacks may have active areas that are, at a minimum, substantially equal to and not smaller than the footprint of the ASICs.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The disclosure also provides support for an electro-acoustic module, comprising: an acoustic stack, and at least one application-specific integrated circuit (ASIC) electrically coupled to the acoustic stack by an interconnect having a fan-out architecture, wherein the electro-acoustic module has an active aperture substantially equal to an overall size of the electro-acoustic module in at least one or an azimuth and an elevation direction. In a first example of the system, the interconnect is a multi-layer interposer, and wherein the interconnect includes input/output (I/O) connections located along an underside of the interconnect, opposite of the acoustic stack. In a second example of the system, optionally including the first example, the interconnect is positioned between the acoustic stack and the at least one ASIC along a propagation direction of the electro-acoustic module, and wherein each layer of a plurality of layers of the interconnect has a bump pitch that is different from adjacent layers. In a third example of the system, optionally including one or both of the first and second examples, the bump pitch of each of the plurality of layers decreases in a direction from the acoustic stack to the at least one ASIC. In a fourth example of the system, optionally including one or more or each of the first through third examples, an element pitch of the acoustic stack is larger than ASIC bump pitch of the at least one ASIC. In a fifth example of the system, optionally including one or more or each of the first through fourth examples, the interconnect has bumps protruding outwards from a top layer of the interconnect, the bumps of the top layer having a first pitch corresponding to an element pitch of the acoustic stack, and bumps protruding outwards from a bottom layer of the interconnect, the bumps of the bottom layer having a second pitch corresponding to an ASIC bump pitch of the at least one ASIC. In a sixth example of the system, optionally including one or more or each of the first through fifth examples, the bottom layer of the interconnect further includes I/O connections arranged peripheral to the bumps of the bottom layer having the second pitch. In a seventh example of the system, optionally including one or more or each of the first through sixth examples, the electro-acoustic module is manufactured having a chip-scale package including the acoustic stack, the at least one ASIC, and the interconnect.

The disclosure also provides support for a method for manufacturing a line of ultrasound probes, the method comprising: obtaining at least one first application-specific integrated circuit (ASIC) having a first area along a plane perpendicular to a propagation direction of a first acoustic stack, and coupling a first interconnect of a plurality of interconnects to the at least one first ASIC, the plurality of interconnects having different fan-out architectures, wherein the first interconnect has a footprint corresponding to a first active aperture of a first ultrasound probe, and wherein the first active aperture of the first ultrasound probe is larger than the first area of the at least one first ASIC. In a first example of the method, the method further comprises: coupling a second interconnect of the plurality of interconnects to at least one second ASIC, the second interconnect selected based on a second active aperture of a second ultrasound probe, the second active aperture larger than the first active aperture, and wherein a second area of the at least one second ASIC is equal to the first area of the at least one first ASIC, and the at least one second ASIC and the at least one first ASIC are of a common type. In a second example of the method, optionally including the first example, the first ultrasound probe is of a different probe type than the second ultrasound probe. In a third example of the method, optionally including one or both of the first and second examples, an acoustic stack of the first ultrasound probe is electrically coupled to the at least one first ASIC by arranging interposer bumps of an interposer of the acoustic stack in face-sharing contact with interconnect bumps of the first interconnect protruding from a top layer of the first interconnect, and arranging interconnect bumps protruding from a bottom layer of the first interconnect in face-sharing contact with ASIC bumps of the at least one first ASIC. In a fourth example of the method, optionally including one or more or each of the first through third examples, the method further comprises: forming the first ultrasound probe as a chip-scale package (CSP) by attaching an acoustic stack of the first ultrasound probe and the at least one first ASIC to the first interconnect using one or more of thermocompression bonding, soldering, conductive epoxy, and ultrasonic bonding. In a fifth example of the method, optionally including one or more or each of the first through fourth examples, an assembly formed of the first interconnect coupled to the at least one first ASIC includes input/output (I/O) connections routed to a bottom side of the assembly.

The disclosure also provides support for a plurality of ultrasound probes, comprising: active apertures of varying sizes amongst the plurality of ultrasound probes, wherein the plurality of ultrasound probes each incorporate at least one ASIC of a common size and type and an interconnect having a fan-out architecture, the at least one ASIC having a footprint equal to or less than a smallest active aperture of the plurality of ultrasound probes. In a first example of the system, a first ultrasound probe of the plurality of ultrasound probes has two or more electro-acoustic modules (EAMs) aligned along a plane perpendicular to a propagation direction of the first ultrasound probe, and wherein a first EAM of the two or more EAMs has a first element pitch that is the same or different from a second element pitch of a second EAM of the two or more EAMs. In a second example of the system, optionally including the first example, the at least one ASIC has through-silicon vias, and wherein additional ASICs are coupled to the at least one ASIC by the through-silicon vias. In a third example of the system, optionally including one or both of the first and second examples, a second ultrasound probe of the plurality of ultrasound probes includes a thermal substrate coupled to a bottom face of the at least one ASIC. In a fourth example of the system, optionally including one or more or each of the first through third examples, a third ultrasound probe of the plurality of ultrasound probes has one or more EAMs are coupled to a common thermal substrate, each of the one or more EAMs including the at least one ASIC, and wherein the common thermal substrate has electronic islands spaced apart by the footprint of the at least one ASIC, the electronic islands configured to provide electrical continuity through the common thermal substrate. In a fifth example of the system, optionally including one or more or each of the first through fourth examples, a fourth ultrasound probe of the plurality of ultrasound probes includes an EAM with the interconnect having the fan-out architecture, peripheral layers, the peripheral layers surrounding a perimeter of the at least one ASIC, and a bottom layer extending across a bottom of the EAM, the bottom layer enclosing the at least one ASIC within the interconnect and having I/O connections protruding outwards therefrom.

In another representation, a method for assembling an electro-acoustic module for a transducer probe includes electrically coupling an acoustic stack to at least one application-specific integrated circuit (ASIC) via an interconnect having a fan-out architecture, wherein an overall aperture of the transducer probe, the overall aperture defined along a plane perpendicular to a propagation direction of the transducer probe, substantially equal to an active aperture of the transducer probe. In yet another representation, a transducer array includes one or more electro-acoustic modules (EAMs) arranged along a common plane, each of the one or more EAMs including an interconnect having a fan-out architecture with input/output (I/O) connections of the interconnect located opposite of an acoustic stack, wherein an interelement kerf width is substantially equal to an intermodule kerf width of the transducer array

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An electro-acoustic module, comprising:
an acoustic stack; and
at least one application-specific integrated circuit (ASIC) electrically coupled to the acoustic stack by an interconnect having a fan-out architecture;
wherein the electro-acoustic module has an active aperture substantially equal to an overall size of the electro-acoustic module in at least one or an azimuth and an elevation direction.

2. The electro-acoustic module of claim 1, wherein the interconnect is a multi-layer interposer, and wherein the interconnect includes input/output (I/O) connections located along an underside of the interconnect, opposite of the acoustic stack.

3. The electro-acoustic module of claim 1, wherein the interconnect is positioned between the acoustic stack and the at least one ASIC along a propagation direction of the electro-acoustic module, and wherein each layer of a plurality of layers of the interconnect has a bump pitch that is different from adjacent layers.

4. The electro-acoustic module of claim 3, wherein the bump pitch of each of the plurality of layers decreases in a direction from the acoustic stack to the at least one ASIC.

5. The electro-acoustic module of claim 1, wherein an element pitch of the acoustic stack is larger than ASIC bump pitch of the at least one ASIC.

6. The electro-acoustic module of claim 1, wherein the interconnect has bumps protruding outwards from a top layer of the interconnect, the bumps of the top layer having a first pitch corresponding to an element pitch of the acoustic stack, and bumps protruding outwards from a bottom layer of the interconnect, the bumps of the bottom layer having a second pitch corresponding to an ASIC bump pitch of the at least one ASIC.

7. The electro-acoustic module of claim 6, wherein the bottom layer of the interconnect further includes I/O connections arranged peripheral to the bumps of the bottom layer having the second pitch.

8. The electro-acoustic module of claim 1, wherein the electro-acoustic module is manufactured having a chip-scale package including the acoustic stack, the at least one ASIC, and the interconnect.

9. A method for manufacturing a line of ultrasound probes, the method comprising:
obtaining at least one first application-specific integrated circuit (ASIC) having a first area along a plane perpendicular to a propagation direction of a first acoustic stack; and
coupling a first interconnect of a plurality of interconnects to the at least one first ASIC, the plurality of interconnects having different fan-out architectures, wherein the first interconnect has a footprint corresponding to a first active aperture of a first ultrasound probe, and wherein the first active aperture of the first ultrasound probe is larger than the first area of the at least one first ASIC.

10. The method of claim 9, further comprising coupling a second interconnect of the plurality of interconnects to at least one second ASIC, the second interconnect selected based on a second active aperture of a second ultrasound probe, the second active aperture larger than the first active aperture, and wherein a second area of the at least one second ASIC is equal to the first area of the at least one first ASIC, and the at least one second ASIC and the at least one first ASIC are of a common type.

11. The method of claim 10, wherein the first ultrasound probe is of a different probe type than the second ultrasound probe.

12. The method of claim 9, wherein an acoustic stack of the first ultrasound probe is electrically coupled to the at least one first ASIC by arranging interposer bumps of an interposer of the acoustic stack in face-sharing contact with interconnect bumps of the first interconnect protruding from a top layer of the first interconnect, and arranging interconnect bumps protruding from a bottom layer of the first interconnect in face-sharing contact with ASIC bumps of the at least one first ASIC.

13. The method of claim 9, further comprising forming the first ultrasound probe as a chip-scale package by attaching an acoustic stack of the first ultrasound probe and the at least one first ASIC to the first interconnect using one or more of thermocompression bonding, soldering, conductive epoxy, and ultrasonic bonding.

14. The method of claim 9, wherein an assembly formed of the first interconnect coupled to the at least one first ASIC includes input/output (I/O) connections routed to a bottom side of the assembly.

15. A plurality of ultrasound probes, comprising:
active apertures of varying sizes amongst the plurality of ultrasound probes, wherein the plurality of ultrasound probes each incorporate at least one ASIC of a common size and type and an interconnect having a fan-out architecture, the at least one ASIC having a footprint equal to or less than a smallest active aperture of the plurality of ultrasound probes.

16. The plurality of ultrasound probes of claim 15, wherein a first ultrasound probe of the plurality of ultrasound probes has two or more electro-acoustic modules (EAMs) aligned along a plane perpendicular to a propagation direction of the first ultrasound probe, and wherein a first EAM of the two or more EAMs has a first element pitch that is the same or different from a second element pitch of a second EAM of the two or more EAMs.

17. The plurality of ultrasound probes of claim 15, wherein the at least one ASIC has through-silicon vias, and wherein additional ASICs are coupled to the at least one ASIC by the through-silicon vias.

18. The plurality of ultrasound probes of claim 15, wherein a second ultrasound probe of the plurality of ultrasound probes includes a thermal substrate coupled to a bottom face of the at least one ASIC.

19. The plurality of ultrasound probes of claim 15, wherein a third ultrasound probe of the plurality of ultrasound probes has one or more EAMs are coupled to a common thermal substrate, each of the one or more EAMs including the at least one ASIC, and wherein the common thermal substrate has electronic islands spaced apart by the footprint of the at least one ASIC, the electronic islands configured to provide electrical continuity through the common thermal substrate.

20. The plurality of ultrasound probes of claim 15, wherein a fourth ultrasound probe of the plurality of ultrasound probes includes an EAM with the interconnect having the fan-out architecture, peripheral layers, the peripheral layers surrounding a perimeter of the at least one ASIC, and a bottom layer extending across a bottom of the EAM, the bottom layer enclosing the at least one ASIC within the interconnect and having I/O connections protruding outwards therefrom.
